(19) United States
(12) Patent Application Publication
(10) Pub. No.: US 2022/0186308 A1
(43) Pub. Date: Jun. 16, 2022

(54) METHODS FOR SEQUENCING NUCLEIC ACID MOLECULES

(71) Applicant: 13.8, Inc., Palo Alto, CA (US)

(72) Inventors: Christina FAN, San Jose, CA (US); Stephen P.A. FODOR, Palo Alto, CA (US)

(21) Appl. No.: 17/683,975

(22) Filed: Mar. 1, 2022

Related U.S. Application Data
(63) Continuation of application No. PCT/US20/49075, filed on Sep. 2, 2020.
(60) Provisional application No. 62/895,375, filed on Sep. 3, 2019.

(51) Int. Cl. C12Q 1/6869; C12N 15/10

(52) U.S. Cl. CPC C12Q 1/6869; C12N 15/1075

(57) ABSTRACT

Provided herein are methods, compositions, and kits for sequencing nucleic acid molecules of a sample in 3 dimensions (e.g., 3D sequencing).

### CROSS-REFERENCE

This application is a bypass continuation application of PCT/US2020/049075, filed Sep. 2, 2020, which claims priority to U.S. Provisional Application No. 62/895,375, filed Sep. 3, 2019, which applications are incorporated by reference herein in their entirety for all purposes.

### SUMMARY

Provided herein are methods, compositions and kits for analyzing and/or sequencing nucleic acid molecules of a sample (e.g., a biological sample).

In various aspects, the present disclosure provides a method of forming a three dimensional (3D) sequencing substrate comprising: (a) amplifying a plurality of nucleic acid molecules from a sample in a plurality of partitions, wherein a partition of the plurality of partitions comprises a nucleic acid molecule from the plurality of nucleic acid molecules and a substrate, and wherein amplification couples the nucleic acid molecule from the plurality of nucleic acid molecules or an amplicon thereof to the substrate; and (b) forming a three dimensional (3D) sequencing substrate from the plurality of partitions.

In various aspects, the present disclosure provides a method of forming a three dimensional sequencing substrate comprising: (a) distributing a plurality of nucleic acid molecules of a sample into a plurality of partitions, wherein a partition of the plurality of partitions comprises a nucleic acid molecule of the plurality of nucleic acid molecules and a substrate of a plurality of substrates; (b) coupling the nucleic acid molecule of the plurality of nucleic acid molecules to the substrate of the plurality of substrates in the partition of the plurality of partitions to form a substrate conjugate of a plurality of substrate conjugates, thereby generating the plurality of substrate conjugates in the plurality of partitions; and (c) forming a three dimensional sequencing substrate from the plurality of partitions.

In various aspects, the present disclosure provides a method of sequencing a plurality of nucleic acid molecules of a sample, the method comprising: (a) forming a three dimensional (3D) sequencing substrate from a plurality of partitions, wherein a partition of the plurality of partitions comprises a substrate conjugate, and the substrate conjugate comprises a nucleic acid molecule of the plurality of nucleic acid molecules of the sample coupled to a substrate; and (b) sequencing the plurality of nucleic acid molecules in the three dimensional sequencing substrate.

In various aspects, the present disclosure provides a method of sequencing a plurality of nucleic acid molecules of a sample, the method comprising: (a) distributing the plurality of nucleic acid molecules of the sample into a plurality of partitions, wherein a partition of the plurality of partitions comprises a nucleic acid molecule of the plurality of nucleic acid molecules and a substrate of a plurality of substrates; (b) coupling the nucleic acid molecule of the plurality of nucleic acid molecules to the substrate of the plurality of substrates in the partition of the plurality of partitions to form a substrate conjugate of a plurality of substrate conjugates, thereby generating the plurality of substrate conjugates in the plurality of partitions; (c) forming a three dimensional sequencing substrate from the plurality of partitions; and (d) sequencing the plurality of nucleic acid molecules in the three dimensional (3D) sequencing substrate.

In various aspects, the present disclosure provides a method of identifying a plurality of nucleic acid molecules of a sample, the method comprising: (a) coupling the plurality of nucleic acid molecules to a substrate to produce a plurality of coupled nucleic acid molecules; (b) partitioning the plurality of coupled nucleic acid molecules into a plurality of partitions such that each partition comprises a nucleic acid molecule coupled to the substrate; (c) forming a three dimensional (3D) sequencing substrate from the plurality of partitions; and (d) sequencing the plurality of coupled nucleic acid molecules, thereby identifying the plurality of nucleic acid molecules of the sample. In some aspects, such method can further comprise, prior to (a), (b), or (c), amplifying the plurality of nucleic acid molecules in the plurality of partitions. In some aspects, amplification comprises thermal cycling amplification or isothermal amplification. In some aspects, the nucleic acid molecule or an amplicon thereof is coupled to the substrate using bioconjugation chemistry or click chemistry. In some aspects, the nucleic acid molecule or an amplicon thereof is coupled to the substrate using a PCR primer comprising a modification at the 5′-end. In some aspects, the modification at the 5′-end comprises an acrydite moiety. In some aspects, the plurality of partitions comprises a plurality of droplets. In some aspects, the plurality of droplets comprises a plurality of emulsion droplets. In some aspects, the substrate comprises a polymer. In some aspects, the polymer comprises an agarose, a polyacrylamide, a UV-curable polymer, a PEG based hydrogel, or a combination thereof. In some aspects, the substrate conjugate is an emulsion bead-nucleic acid conjugate, a polymer bead-nucleic acid conjugate, or a combination thereof. In some aspects, the plurality of partitions are attached to each other, thereby forming the 3D sequencing substrate. In some aspects, plurality of partitions are attached to each other by addition of substrate to the plurality of partitions, by an elevation of temperature, or a combination thereof. In some aspects, the 3D sequencing substrate is a gel matrix. In some aspects, the sequencing is conducted in a vessel. In some aspects, the vessel is a sphere, a cylinder, a cube, a cone, a hexagon, a prism, or any combination or variation thereof. In some aspects, the vessel is a tube, a syringe, a micro-container, a spin column, a flow cell, or a combination thereof. In some aspects, the 3D sequencing substrate has the same shape as the vessel. In some aspects, the 3D sequencing substrate is formed by centrifugation. In some aspects, the 3D sequencing substrate has a volume from about 1 μL to about 1000 μL. In some aspects, the 3D sequencing substrate comprises from about 10³ to about 10¹⁵ partitions. In some aspects, the plurality of partitions of the 3D sequencing substrate assemble in a cubic closest packed unit cell. In some aspects, each partition of a plurality of partitions has an average diameter of about 1 μm to about 50 μm. In some aspects, sequencing is performed inside the 3D sequencing substrate. In some aspects, the 3D sequencing substrate is transparent. In some aspects, the sequencing reaction in the 3D sequencing substrate is monitored in 3D using 3D imaging. In some aspects, the 3D imaging comprises confocal microscopy, super-resolution confocal microscopy, multiphoton microscopy, or lightsheet microscopy, or a combination thereof. In some aspects, the substrate conjugates in the 3D sequencing substrate are detected via 3D imaging as spots of nucleic acid molecules. In some aspects, one or more nucleic acid molecules of the plurality of nucleic acid molecules are detected in no more than about 50%, no more than about 10%, no more than about 5%, or no more than about 1% of the plurality of partitions of the 3D sequencing substrate. In some aspects, sequencing comprises pyrosequencing, sequencing by synthesis, sequencing by ligation, sequencing by hybridization, sequencing by degradation, sequencing by detection of local pH changes, sequencing by denaturation, or any combination thereof.

In various aspects, the present disclosure provides a composition comprising (a) a three dimensional (3D) sequencing substrate; (b) a plurality of nucleic acid molecules; and (c) sequencing reagents. In some aspects, the 3D sequencing substrate comprises a plurality of partitions. In some aspects, a partition of the plurality of partitions comprises a nucleic acid molecule of the plurality of nucleic acid molecules. In some aspects, the nucleic acid molecule of the plurality of nucleic acid molecules is coupled to the substrate inside the partition. In some aspects, the plurality of partitions is a plurality of droplets. In some aspects, the substrate comprises a polymer. In some aspects, the polymer comprises an agarose, a polyacrylamide, a UV-curable polymer, a PEG based hydrogel, or a combination thereof. In some aspects, the nucleic acid molecule is coupled to the substrate using bioconjugation chemistry or click chemistry. In some aspects, the nucleic acid molecule or an amplicon thereof is coupled to the substrate using a PCR primer comprising a modification at the 5′-end. In some aspects, the modification at the 5′-end comprises an acrydite moiety. In some aspects, the plurality of partitions forming the 3D sequencing substrate are attached to each other. In some aspects, the plurality of partitions are attached to each other by addition of substrate to the plurality of partitions, by an elevation of temperature, or a combination thereof.

In various aspects, the present disclosure provides a kit for nucleic acid sequence identification of a sample comprising: (a) substrate reagents for forming a three dimensional (3D) sequencing substrate; (b) amplification reagents; (c) sequencing reagents; and (d) instructions that direct a user to use the substrate reagents, the amplification reagents, and the sequencing reagents for nucleic acid sequence identification of the sample in the 3D sequencing substrate. In some aspects, the 3D sequencing substrate has the same shape as the part of the vessel comprising the 3D sequencing substrate. In some aspects, the vessel further comprises a vessel in which to form the 3D sequencing substrate. In some aspects, the vessel is a tube, a syringe, a micro-container, a spin column, a flow cell, or a combination thereof. In some aspects, the kit comprises a plurality of nucleic acid molecules of the sample, and wherein the plurality of nucleic acid molecules is coupled to the 3D sequencing substrate, thereby forming a plurality of substrate conjugates in the 3D sequencing substrate. In some aspects, the substrate reagents comprise agarose, a polymer, or a hydrogel. In some aspects, the 3D sequencing substrate is transparent. In some aspects, the sequencing reaction using the sequencing reagents in the 3D sequencing substrate is monitored in 3D using 3D imaging. In some aspects, 3D imaging comprises confocal microscopy, super-resolution confocal microscopy, multiphoton microscopy, or lightsheet microscopy, or a combination thereof. In some aspects, the substrate conjugates in the 3D sequencing substrate are detected as spots of nucleic acid molecules. In some aspects, the plurality of substrate conjugates is a plurality of emulsion bead-nucleic acid conjugates, a plurality of polymer bead-nucleic acid conjugates, or a combination thereof. In some aspects, the 3D sequencing substrate has a volume of about 1 μL to about 1000 μL. In some aspects, the sequencing comprises pyrosequencing, sequencing by synthesis, sequencing by ligation, sequencing by hybridization, sequencing by degradation, sequencing by detection of local pH changes, sequencing by denaturation, or any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Next-Generation Sequencing (NGS) techniques, such as Illumina, 454 sequencing, Ion Torrent, PacBio, Helicos, etc., generally utilize single nucleic acid molecules or clones of single nucleic acid molecules to be sequenced that are positioned on a single plane. The sequencing substrate (e.g., a plane or a surface) can be housed inside a flow cell in which chemical (e.g., sequencing) reaction can occur. Due to the two-dimensional (2D) nature of the sequencing substrate, most reactants in the volume (e.g., of a reaction solution) above such plane or surface to which the nucleic acid molecules may be attached to, may not participate in the sequencing reaction and thus may be wasted. Additionally, positioning single molecules or clones of single molecules on a planar (e.g., 2D) substrate requires specialized consumables. For instance, for 454 and Ion Torrent, clones of nucleic acid molecules in the forms of sequencing beads (e.g., polymer beads having nucleic acid molecules attached to their surface) are loaded onto microwell arrays. In order to fill the microwell arrays to saturation, the number of beads loaded may be in excess of the number of wells, and thus a large portion of the bead library may not be sequenced. The process to distribute the molecules across the planar substrate may add time and labor to the workflow, and hence may result in loss of sample (e.g., a biological sample). Thus, there exists a need for more resource-efficient and faster sequencing methods, particularly in areas where a high number of samples needs to be analyzed with fast turn-around times.

Contemplated herein are methods, compositions, and kits for the sequencing of nucleic acid molecules of a sample (e.g., a biological sample) in three dimensions (e.g., 3D sequencing). Thus, in some aspects, sequencing of nucleic acid molecules using the herein described methods, compositions, and kits may provide a significantly higher density of information (e.g., sequence information obtained per reaction volume) compared to conventional 2D sequencing methods. For example, all or nearly all reagents that pass through the sequencing substrate participate in sequencing reactions and only minimal amounts of reagents (e.g., sequencing reagents such as enzyme, nucleotides, etc.) may be wasted (e.g., those that may not participate in the sequencing reactions).

Additional advantages provided by the methods, compositions, and kits of the present disclosure include but are not limited to: (i) clonal amplification may be performed using simple laboratory equipment and without the need for specialized instrumentation (e.g., specialized flow cell instrumentation); (ii) fast and easy-to-use preparation and procedural protocols; (iii) library amplification and clonal amplification may be combined into a single reaction, resulting in faster turn-around while requiring less labor and resources; (iv) all or nearly all sample nucleic acid molecules going into the library amplification can be sequenced, thereby avoiding loss of template or sample material (e.g., particularly important if only very limited amounts of sample material is available such as in the case of cell-free DNA, biopsies, etc.); (v) the use of 3D sequencing substrate materials (e.g., hydrogel) may be compatible with sequencing chemistries that can provide longer read length and faster reaction time; (vi) individual samples (e.g., clinical patient samples) may be analyzed separately and/or in parallel due to the small sample volumes that the herein described methods can be used with, and thus the individual samples may not need to be pooled, avoiding/minimizing potential cross contamination between samples; and (vi) the sequencing reaction(s) may be monitored in 3D (e.g., by using transparent substrate material), which may yield much higher information density (e.g., per reagent consumption, per volume of sequencing substrate, per amount of sample, etc.).

The methods, compositions, and kits of the present disclosure can comprise coupling a plurality of nucleic acid molecules of a sample (e.g., a biological sample) to a substrate. In some cases, the plurality of nucleic acid molecules of the sample may be distributed into a plurality of partitions (e.g., droplets and/or wells). Distribution of the nucleic acid molecules into the plurality of partitions may occur prior to or after coupling of the plurality of nucleic acid molecules to the substrate. The nucleic acid molecules can comprise DNA such as chromosomal DNA (e.g., cDNA), circulating DNA such as circulating tumor DNA (ctDNA), and RNA such as mRNAs, shRNAs, siRNA, etc.

The nucleic acid molecules may be obtained from a sample. The sample may be a biological sample. The biological sample may be from a mammal such as a human or a rodent as further described elsewhere herein. The terms “biological sample” and “sample,” as used herein, can be used interchangeably and generally refer to materials obtained from or derived from a subject (e.g., a human). A biological sample can include sections of tissues such as biopsy and autopsy samples, and frozen sections taken for histological purposes. Such samples can include bodily fluids such as blood and blood fractions or products (e.g., serum, plasma, platelets, red blood cells, and the like), feces and feces fractions or products (e.g., fecal water, such as but not limited to fecal water separated from other fecal components and solids by methods such as centrifugation and filtration), sputum, tissue, cultured cells (e.g., primary cultures, explants, and transformed cells), stool, urine, synovial fluid, joint tissue, synovial tissue, synoviocytes, fibroblast-like synoviocytes, macrophage-like synoviocytes, immune cells, hematopoietic cells, fibroblasts, macrophages, dendritic cells, T-cells, etc. A sample can be obtained from a eukaryotic organism, such as a mammal such as a primate e.g., chimpanzee or human, cow, dog; cat, a rodent, e.g., guinea pig, rat, mouse; rabbit, or a bird, reptile, or fish.

The methods, compositions, and kits of the present disclosure can comprise a substrate. A substrate can comprise one or more different molecules. A substrate may comprise one or more polymers and/or molecular building blocks (e.g., monomers) that can form one or more polymers. The substrate can comprise molecules such as monomeric molecules capable of forming polymers, including but not limited to, carbohydrates such as galactose (e.g., D- or L-galactose), 3,6-anhydro-L-galactopyranose, acrylamide, acrydite, etc. A substrate can comprise polymeric molecules, such as agarose, modified agarose polymers (e.g., agarose polymers modified to comprise functional groups for further functionalization or coupling to nucleic acid molecules), polyacrylamide, modified polyacrylamide (e.g., polyacrylamide polymers modified to comprise functional groups for further functionalization or coupling to nucleic acid molecules), etc. A substrate can comprise monomeric and/or polymeric molecules capable of forming hydrogels such as polyethylene glycol (PEG)-based hydrogels.

A substrate can be a homogenous substrate. Such substrate can be functionalized to allow coupling of nucleic acid molecules to the substrate. The substrate can be in a vessel (e.g., a tube, a syringe, a micro-container, a spin column, a flow cell, or a combination thereof). The substrate can be liquid or solid, and may have a certain viscosity. The viscosity of a substrate can be controlled using various external stimuli such as temperature, radiation (e.g., UV light), chemical compounds, etc. For example, the present disclosure provides agarose-based polymer substrate which viscosity can be altered or controlled using different temperatures. In some cases, an agarose-based polymer substrate can be liquid (e.g., have an increased viscosity) at temperatures >50° C. and solid (e.g., have a decreased viscosity) at temperatures <50° C. Altering or controlling the viscosity of a substrate can be used in the herein described methods for, e.g., allowing amplification and/or sequencing reactions to occur, and the formation of 3D sequencing substrates by temporarily melting the substrate of a 3D sequencing substrate allowing partitions (e.g., droplets) to attach to each other.

A substrate of the present disclosure can be used to form one or more beads. Such beads can be polymer beads. Beads can be formed using any technique suitable for generating such beads. The beads used in combination with the herein described methods, compositions, and kits can be functionalized (e.g., surface-functionalized). Such functionalization can include functional groups suitable for coupling nucleic acid molecules onto the beads. Such functional groups can include reactive moieties capable of reacting with certain other moieties or functional groups. For example, amplification and coupling of amplicons of a sample nucleic acid to a polyacrylamide-based substrate such as a polyacrylamide-based bead can occur using primer molecules comprising an acrydite moiety at the 5′-end. In the presence of radical initiator molecules (e.g., TEMED), the amplified nucleic acid molecules can be attached or localized to the substrate. As described herein, such amplification and coupling reactions can be performed in a vessel comprising the substrate. Amplification and coupling reactions can be conducted in partitions. Such partitions can be physically separated from each other, e.g., allowing the amplification of as few as one nucleic acid molecule in one partition, and subsequent coupling of the amplicons to the substrate within that partition (e.g., a droplet such as an emulsion droplet, or a well).

Coupling reactions used herein to couple a nucleic acid molecule to a substrate (e.g., a bead) can form covalent and/or non-covalent bonds between, e.g., a nucleic acid and a substrate. Coupling reactions can comprise bioconjugation chemistries and/or click chemistries. Bioconjugation chemistry as described herein can refer to any chemical reaction that links, couples, or attaches a nucleic acid molecule of a sample with a substrate. Such bioconjugation chemistry can comprise biological interactions (e.g., biotin/strepdavidin interactions) and/or bioorthogonal reactions. In other case, coupling or attachment of nucleic acid molecules can be performed using click chemistry. Click chemistry can comprise any type of click reaction suitable for coupling nucleic acid molecules to substrates. Examples of click chemistry reactions (or short “click reactions”) that can be used in combination with the herein described methods and compositions include, but are not limited to, transition-metal catalyzed or strain-promoted azide-alkyne cycloadditions (e.g., Huisgen azide-alkyne 1,3-dipolar cycloaddition, copper-catalyzed azide-alkyne cycloaddition (CuAAC), strain-promoted alkyne-azide cycloaddition, and/or ruthenium-catalyzed azide-alkyne cycloaddition (RuAAC)), Diels-Alder reactions such as inverse-electron demand Diels-Alder reaction (e.g., tetrazine-trans-cyclooctene reactions), or photo-click reactions (e.g., alkene-tetrazole photoreactions). In some embodiments, nucleic acid molecules may be localized to the substrate via non-bonding interactions. For example, a dense gel matrix may restrict the movement of a nucleic acid molecule, thereby confining the nucleic acid molecule in space.

The herein described methods, compositions, and kits can allow any sequencing chemistry to be carried out in the substrate. Such sequencing chemistries include, but are not limited to, pyrosequencing, sequencing by synthesis, sequencing by ligation, sequencing by hybridization, sequencing by degradation (e.g., by exonuclease), sequencing by detection of local pH changes (e.g., due to release of protons from polymerase extension such as by pH sensitive dyes), and sequencing by denaturation. In particular, chemistries that involve signal molecules that can be released during the reaction can be used, e.g., as the substrate (e.g., hydrogel) retards diffusion (e.g., when lowering the temperature). Examples include pyrosequencing (e.g., as in 454 sequencing), or sequencing by synthesis with fluorophore attached to 5′ phosphate (as in PacBio, also referred to herein as “Single Molecule, Real-Time” (SMRT) sequencing). Compared to sequencing chemistry using reversible terminators (e.g., as used in Illumina sequencing), the chemistries utilized and described herein can use natural nucleotides and polymerases and thus may leave little scar on the growing DNA strand, which can result in faster sequencing speed and longer read-length.

As described herein, the substrate used in combination with the herein described methods, compositions, and kits can form a three-dimensional (3D) sequencing substrate. Such 3D sequencing substrate can be generated by packing partitions (e.g., emulsion droplets), beads (e.g., polymer beads, hydrogel beads, etc.), or partitions containing such beads into a 3D volume. Such a 3D volume can have various sizes and shapes. A 3D volume can be a vessel having a certain size and shape. The shape of a vessel or the shape of a part of a vessel can be a sphere, a cylinder, a cube, a cone, a hexagon, a prism, or any combination or variation thereof. A vessel can be a tube, a syringe, a micro-container, a spin column, a flow cell, or a combination thereof. Thus, a 3D sequencing substrate can take various shapes and forms, such as a sphere, a cylinder, a cube, a cone, a hexagon, a prism, or any combination or variation thereof. The 3D sequencing substrate can have the same shape as a vessel or part of a vessel. In one example, a 3D sequencing substrate has the same shape as a vessel or part of a vessel by adding the 3D sequencing substrate (e.g., a liquid 3D sequencing substrate) to the vessel. In another example, a 3D sequencing substrate has the same shape as a vessel or part of a vessel by packing the vessel or part of the vessel with emulsion droplets or beads comprising the substrate. The emulsion droplets or beads of the 3D sequencing substrate can have spherical shapes and thus packing a 3D volume with these spherical droplets and/or beads may result in various orders. The droplets or beads of the 3D sequencing substrate may assemble in various packing orders such as cubic close packing or hexagonal close packing.

A sequencing reaction (e.g., pyrosequencing, sequencing by synthesis, sequencing by ligation, sequencing by hybridization, sequencing by degradation, sequencing by detection of local pH changes, or sequencing by denaturation) may be performed on a nucleic acid sequence after formation of the 3D substrate comprising the nucleic acid sequence. For example, sequencing by synthesis, which is a cyclic process, may be performed on a 3D substrate by passing reagents of each sequencing step through the vessel. In some embodiments, the vessel may be a tube, a syringe, a micro-container, a spin column, or a flow cell. In some embodiments, the vessel may have a valve controlling reagent flow out of the vessel. The vessel may be incubated, during which time the sequencing reaction may occur. Following, the sequencing reaction, the substrate may be imaged in three dimensions. For example, the entire vessel volume may be imaged in three dimensions. Following imaging, the vessel may be washed and the repeating the sequencing reaction and imaging cycle.

A 3D sequencing substrate as described herein can be generated using various methods. These methods can include centrifugation, filtration, etc. In an example, a 3D sequencing substrate is generated from a plurality of emulsion droplets, e.g., those comprising polymer or hydrogel beads, by separating the droplets from the oil-phase (e.g., via washing with alcohol, detergent, etc.) and subsequent spinning thereby packing the polymer or hydrogel beads in the 3D volume generating a 3D sequencing substrate. A 3D sequencing substrate as described herein can be further modified, e.g., by physically attaching the polymer or hydrogel beads to each other (e.g., by increasing the temperature and allowing the beads to slightly melt and stick together, and/or by adding substrate to the beads resulting in inter-bead attachments (e.g., through crosslinking of beads, etc.)). In some cases, the attachments of beads, or any other partitions, to one another can form a 3D sequencing matrix (e.g., a polymer matrix or a hydrogel matrix). In some cases, a 3D sequencing substrate as described herein can be further modified, e.g., by physically attaching the polymer or hydrogel beads to each other by resuspending the packed polymer or hydrogel beads in a small amount of additional solution (e.g., molten agarose, a polyacrylamide solution, etc.), thereby holding together the 3D sequencing matrix (e.g., a polymer matric or a hydrogel matrix).

A 3D sequencing substrate can have various volumes. The volume of a 3D sequencing substrate can be from about 1 μL to about 10 mL. The volume of a 3D sequencing substrate can be from about 10 μL to about 1 mL. The volume of a 3D sequencing substrate can be from about 100 μL to about 1000 μL. The volume of a 3D sequencing substrate can be from about 50 μL to about 500 μL. The volume of a 3D sequencing substrate can be at least about 1 μL. The volume of a 3D sequencing substrate can be at least about 10 μL. The volume of a 3D sequencing substrate can be at least about 50 μL. The volume of a 3D sequencing substrate can be at least about 100 μL. The volume of a 3D sequencing substrate can be at least about 200 μL. The volume of a 3D sequencing substrate can be at least about 500 μL. The volume of a 3D sequencing substrate can be at least about 1000 μL.

The volume of a 3D sequencing substrate can be from about 1 μL, 10 μL, 50 μL, 100 μL, 150 μL, 200 μL, 250 μL, 500 μL, 750 μL, or 1 mL.

A 3D sequencing substrate can comprise from about 10² to about 10²⁰ partitions (e.g., droplets and/or beads). A 3D sequencing substrate can comprise from about 10³ to about 10¹⁸ partitions (e.g., droplets and/or beads). A 3D sequencing substrate can comprise from about 10⁴ to about 10¹⁶ partitions (e.g., droplets and/or beads). A 3D sequencing substrate can comprise from about 10⁶ to about 10¹⁴ partitions (e.g., droplets and/or beads). A 3D sequencing substrate can comprise from about 10⁸ to about 10¹² partitions (e.g., droplets and/or beads). A 3D sequencing substrate can comprise at least about 10² partitions (e.g., droplets and/or beads). A 3D sequencing substrate can comprise at least about 10⁴ partitions (e.g., droplets and/or beads). A 3D sequencing substrate can comprise at least about 10⁶ partitions (e.g., droplets and/or beads). A 3D sequencing substrate can comprise at least about 10⁸ partitions (e.g., droplets and/or beads). A 3D sequencing substrate can comprise at least about 10¹⁰ partitions (e.g., droplets and/or beads). A 3D sequencing substrate can comprise at least about 10¹² partitions (e.g., droplets and/or beads). A 3D sequencing substrate can comprise at least about 10¹⁴ partitions (e.g., droplets and/or beads). A 3D sequencing substrate can comprise at least about 10¹⁶ partitions (e.g., droplets and/or beads).

A 3D sequencing substrate can comprise at least about 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰ partitions.

Partitions in a sequencing substrate may have an average diameter of from about 0.01 μm to about 200 μm. Partition size may be selected based on the detection technology to be used. For example, sub-micron partition sizes may be used for super resolution imaging (e.g., for single molecule detection). In some embodiments, the partitions may have an average diameter of from about 0.01 μm to about 0.1 μm, from about 0.1 μm to about 1 μm, from about 1 μm to about 5 μm, from about 5 μm to about 10 μm, from about 10 μm to about 15 μm, from about 15 nm to about 20 μm, from about 20 μm to about 25 μm, from about 25 μm to about 30 μm, from about 30 μm to about 35 μm, from about 35 μm to about 40 μm, from about 40 μm to about 45 nm, from about 45 μm to about 50 μm, from about 50 μm to about 100 μm, from about 100 μm to about 200 μm.

The methods, compositions, and kits of the present disclosure can comprise a 3D sequencing substrate comprising, wherein from about 0.1% to about 99% of partitions comprise at least one nucleic acid molecule of a sample (e.g., a biological sample), resulting in about 0.1% occupancy to about 99% occupancy. A 3D sequencing substrate can have from about 0.1% occupancy to about 95% occupancy. A 3D sequencing substrate can have from about 0.1% occupancy to about 50% occupancy. A 3D sequencing substrate can have from about 0.1% occupancy to about 25% occupancy. A 3D sequencing substrate can have from about 0.1% occupancy to about 10% occupancy. A 3D sequencing substrate can have from about 0.1% occupancy to about 5% occupancy. A 3D sequencing substrate can have from about 5% occupancy to about 95% occupancy. A 3D sequencing substrate can have from about 10% occupancy to about 90% occupancy. A 3D sequencing substrate can have from about 20% occupancy to about 80% occupancy. A 3D sequencing substrate can have from about 30% occupancy to about 70% occupancy. A 3D sequencing substrate can have from about 40% occupancy to about 60% occupancy. A 3D sequencing substrate can have at least about 0.1% occupancy. A 3D sequencing substrate can have at least about 2% occupancy. A 3D sequencing substrate can have at least about 5% occupancy. A 3D sequencing substrate can have at least about 10% occupancy. A 3D sequencing substrate can have at least about 20% occupancy. A 3D sequencing substrate can have at least about 30% occupancy. A 3D sequencing substrate can have at least about 40% occupancy. A 3D sequencing substrate can have at least about 50% occupancy. A 3D sequencing substrate can have at least about 60% occupancy. A 3D sequencing substrate can have at least about 70% occupancy. A 3D sequencing substrate can have at least about 80% occupancy. A 3D sequencing substrate can have at least about 90% occupancy. A 3D sequencing substrate can have at least about 95% occupancy. A 3D sequencing substrate can have at least about 99% occupancy. A 3D sequencing substrate can have no more than about 50% occupancy. A 3D sequencing substrate can have no more than about 40% occupancy. A 3D sequencing substrate can have no more than about 30% occupancy. A 3D sequencing substrate can have no more than about 20% occupancy. A 3D sequencing substrate can have no more than about 10% occupancy. A 3D sequencing substrate can have no more than about 5% occupancy. A 3D sequencing substrate can have no more than about 1% occupancy.

The present disclosure provides 3D sequencing substrates that can have various shapes or forms. A 3D sequencing substrate of this disclosure can be a sphere, a cylinder, a cube, a cone, a hexagon, a prism, or any combination or variation thereof. In cases where the 3D sequencing substrate is located in a container, such as a vessel, the 3D sequencing substrate can have the same shape or form as the container, or part of the container. In an example, a container comprises a 3D sequencing substrate, wherein the part of the container comprising the 3D sequencing substrate is a tube, syringe, or a flow cell.

Thus, the present disclosure provides methods, compositions, and kits that can allow for analysis of various samples. Such samples can be biological samples, e.g., clinical samples from subjects. The methods, compositions, and kits of this disclosure can allow for simple, fast and efficient analysis of such samples for analyzing nucleic acid molecules of that sample. Due to significantly increased information density compared to conventional sequencing methods, the 3D sequencing methods described herein can allow for small sample volumes (e.g., between about 1 μL and 200 μL) to be sufficient for analysis of various samples. One advantage of the herein described methods can be that individual samples (e.g., from individual sources such as clinical patients) may not need to be pooled in order to be analyzed, but instead can be analyzed individually in a simple, fast and efficient manner, avoiding, for example, potential cross contamination between various samples. Moreover, the herein described methods, compositions, and kits can allow for analysis of both single molecules such as single nucleic acid molecules of a sample (e.g., single molecule sequencing), or clonal copies (e.g. in the form of polony (e.g., polymerase colony), cluster, bead etc.) of such molecules, or a combination thereof.

The term “subject,” as used herein, generally refers to a living member of the animal kingdom. The subject may be suffering from or may be suspected of suffering from a disease or disorder. The subject can be a member of a species comprising individuals who naturally suffer from the disease. The subject can be a mammal. Non-limiting examples of mammals can include rodents (e.g., mice and rats), primates (e.g., lemurs, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. The subject can be a human. The subject can be a non-mammalian animal such as a turkey, a duck, or a chicken. The subject can be a farm animal (e.g., pig, goat or cow). The subject can be a living organism suffering from or prone to a disease or condition that can be diagnosed and/or treated using the kits, methods, and systems as provided herein. The subject can provide a biological sample (e.g., a fecal sample or blood sample) which can be collected, transported, stored, cultured and/or analyzed using the kits, methods, devices and systems provided herein. The subject may be a patient being treated or monitored by a healthcare provider (e.g., a primary care physician). Alternatively, the subject may not be a patient.

The term “about,” as used herein in the context of a numerical value or range, generally refers to ±10% of the numerical value or range recited or claimed, unless otherwise specified.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As described herein, the 3D sequencing substrates can be generated or formed in various ways using any suitable technique. Such techniques can include the use of various compounds such as polymers, copolymers, hydrogels, and any functionalized derivatives thereof, e.g., to allow coupling or attachment of molecules (e.g., nucleic acid molecules of a sample, and/or amplicons thereof). Such techniques can further comprise using emulsions for droplet generation, or any other suitable method for generating partitions. Such partitions can be used to amplify sample molecules, e.g., by using thermal cycling amplification (e.g., PCR), isothermal amplification, or any related method for nucleic acid amplification.

### 3D Sequencing Substrate Formation

As described herein, the methods, compositions, and kits of this disclosure can be used in combination with a single molecule (e.g., a nucleic acid molecule of a sample) and/or clones of such single molecules. In embodiments where nucleic acid amplification is used, a nucleic acid (e.g., DNA) molecule from a sample (e.g., from a biological sample) can be clonally amplified, e.g., by rapid droplet digital polymerase chain reaction (PCR) techniques or isothermal amplification techniques. For targeted sequencing, for example, the input is purified genomic DNA. In these cases, target specific primers carry sequencing primer sequences on the 5′ end of the purified genomic DNA. In another example, for de novo sequencing, the input is DNA with adaptors ligated to the DNA molecules.

Droplet generation, as described herein, can comprise a droplet generating device (e.g., a microcapillary array or a microfluidic device) via centrifugation, vortexing with hydrogel beads, or a combination thereof. Additional techniques can be used, such as droplet generation by flow focusing on microfluidic chips. As an example, conventional Illumina or Ion Torrent sequencing can require a library amplification step before clonal amplification, which adds to overall sample preparation time and thus intensifies the workflow. In an embodiment of the present disclosure, these two amplification reactions can be combined into one, allowing for more rapid and easy amplification, and reduce labor and resources needed for sample analysis.

The emulsion post amplification (e.g., PCR) can be transferred to a vessel, such as a spin column or a pipette tip. In cases where beads (e.g., polymer beads) are used in droplets for amplification, washing of beads, packing of beads, gelation into sequencing substrate (e.g., by adding additional polymer, generating a hydrogel, increasing the temperature to allow polymer beads to stick together, etc.), and sequencing reagent exchanges can be all carried out in the same vessel (e.g., spin column or a pipette tip). A vessel comprising a hole and a stopper (e.g., a spin column) may allowing reagents to be added to the top of the vessel and drained from the bottom of the vessel (e.g., using gravitational force, vacuum, or centrifugation). This may facilitate reagent exchange for repeating reaction cycles (e.g., sequencing reaction cycles). Fluid exchange can be performed using pressurized gas and/or vacuum.

As described herein, the 3D sequencing substrate can be a hydrogel. The hydrogel can be optically clear. Such hydrogels can be used in combination with various imaging techniques to, e.g., image the substrate volume, monitor the sequencing reaction(s), etc. Such imaging techniques can include lightsheet imaging, confocal microscopy, super-resolution confocal, or multiphoton imaging, e.g., to image the substrate volume. In some embodiments, an imaging technique (e.g., a 3D imaging technique) comprises fluorescent imaging. TABLE 1 in EXAMPLE 1 shows an exemplary number of positive droplets (or reads) that can be fitted into a volume of 100 μl, assuming 10% of the droplets are positive. For example, droplets with an average diameter of about 15 μm allow for about 4 million reads to be attained. When droplets with an average diameter of about 10 μm are used about 14 million reads can be achieved, and so forth. These numbers of possible reads demonstrate that sample volumes of about 100 μl (or lower) can provide surprisingly high information density. Thus, the number of reads that the 3D sequencing methods described herein provide can be sufficient for various clinical assays to be performed, e.g., enabling clinical assays such as targeted panels, shallow whole genome for non-invasive prenatal testing (NIPT), screening and detection of various diseases and conditions (e.g., cancer screening and detection). Moreover, these analyses can be performed on a single sample basis in rapid and efficient manner.

In an example for nucleic acid analysis of a sample, a plurality of single molecules (e.g., nucleic acid molecules of a sample) are physically separated into individual compartments or partitions. Such partitions can be droplets such as emulsion droplets. A partition such as a droplet can comprise one or more nucleic acid molecules of the plurality of nucleic acid molecules. A partition such as a droplet can comprise at least one nucleic acid molecule of the plurality of nucleic acid molecules. A partition such as a droplet can comprise at most one nucleic acid molecule of the plurality of nucleic acid molecules. Within these partitions, a nucleic acid molecule of the plurality of nucleic acid molecules can be clonally amplified (e.g., using PCR). Thus, a partition such as a droplet can further comprise a set of reagents, wherein such set of reagents can comprise reagents that may be used for, e.g., nucleic acid amplification. A partition can also comprise a substrate, and reagents that can allow coupling a nucleic acid molecule or an amplicon thereof to said substrate. Such substrate can be and/or can form a bead. The substrate can be a polymer and thus can form a polymer bead. As described herein, a nucleic acid molecule can be attached to the substrate using various bioconjugation strategies, click chemistry, etc. Subsequently, the plurality of compartments (e.g., partitions) can be integrated together to form a 3D volume. Thus, a 3D sequencing substrate can be formed by packing partitions and/or emulsion droplets in a volume. The 3D sequencing substrate can be formed by packing polymer beads in a volume, or by packing polymer beads that are formed after solidification in individual emulsion. In some cases, a 3D sequencing substrate as described herein are made by physically attaching the polymer or hydrogel beads to each other by resuspending the packed polymer or hydrogel beads in a small amount of additional solution (e.g., molten agarose, a polyacrylamide solution, etc.), thereby holding together the 3D sequencing matrix (e.g., a polymer matric or a hydrogel matrix). Examples of polymers that can be used as a substrate can include agarose, polyacrylamide, UV curable polymers, PEG based hydrogels, or a combination thereof. Alternatively, the substrate can be formed by numerous individual DNA origami structure(s), wherein each of such structure can carry a single molecule (e.g., nucleic acid molecule) to be sequenced.

In another example for nucleic acid analysis of a sample, a plurality of single molecules (e.g., nucleic acid molecules of a sample) can be first captured inside a substrate (e.g., a polymer, hydrogel, etc.) by, e.g., coupling the single molecules to the substrate as described herein. Such coupling can be performed via probes anchored on the substrate. Such probes can be nucleic acid molecule attached to the substrate that can be used to couple sample nucleic acid molecules to the substrate by nucleic acid hybridization. Once coupled to the substrate, these single molecules can be subsequently locally amplified to form clones. The substrate used in such a method can be solid gel with anchored probes (e.g., nucleic acid molecules such as primers), or gel that can be initially in solution during DNA capture and subsequently solidified.

The substrates used in combination with the herein described methods, compositions, and kits can be compatible with any sequencing chemistry to be carried out in the substrate. Such sequencing chemistries include pyrosequencing, sequencing by synthesis, sequencing by ligation, sequencing by hybridization, sequencing by degradation, sequencing by detection of local pH changes, sequencing by denaturation, or sequencing by monitoring polymerase activity. These sequencing chemistries, conventionally carried out in 2D on a surface, can be carried out in 3D in a volume as described in this disclosure. Moreover, the substrates used herein can allow for various sets of reagents used for amplification, coupling of molecules to the substrate, and sequencing to be incorporated inside the substrate, rather than replenishing such reagents during operation as used in conventional methods. Such reagents can include enzymes (e.g., polymerases), nucleic acid molecules, dNTPs, buffers, functionalized molecules such as functional monomers used to couple nucleic acid molecules to the substrate. As described herein, detection of sequencing reaction(s) can be performed using 3D imaging techniques, such as confocal microscopy, super-resolution confocal, multiphoton imaging, and lightsheet microscopy. In some embodiments, a 3D imaging technique comprises fluorescent imaging. To facilitate detection by imaging, the substrate can be naturally optically transparent, or be cleared to become transparent after DNA capture and before sequencing reactions.

The present disclosure provides methods, compositions, and kits that can be used for 3D sequencing of nucleic acid molecules of various samples (e.g., biological samples such as clinical samples). The methods described herein can comprise various strategies for droplet generation, substrate generation, sequencing, etc.

3D Sequencing and Droplet Generation by Droplet Generating Device using Agarose. The present disclosure provides methods that can allow for 3D sequencing and droplet generation using a droplet generating device and agarose (or functionalized agarose) as a substrate. A droplet generating device may have pores that enable fluid to be dropletized by centrifugation. For example, a droplet generating device may be a microcapillary array, a nozzle, or a microfluidic device (e.g., comprising a T-junction). A droplet generating device may utilize pressure (e.g., air or fluid pressure) or centrifugal force (e.g., centrifugation) to form droplets by forcing the fluid through one or more holes, pores, or channels. In such a method, the dispersion phase can contain molten agarose in addition to reagent sets (e.g., PCR master mix) and a plurality of sample nucleic acid molecules. PCR can be performed. At PCR cycling temperatures (e.g., T>50° C.), agarose can remain liquid, allowing for rapid and efficient PCR amplification. The sample nucleic acid molecules can be attached to the agarose substrate. This may be done by using PCR primers that comprise a modification on the 5′ end, thereby coupling one strand of the PCR product to agarose. Such a modification can comprise an acrydite moiety or any other functional modification. Subsequent to PCR, temperature can be lowered and agarose can become solid (e.g., less viscous). In some embodiments, the agarose may be solidified without amplifying the nucleic acid (e.g., for single molecule sequencing). The oil phase (of the original dispersion phase) can be removed by washing with alcohol and/or detergent, thereby generating a plurality of agarose beads. In some embodiments, the agarose beads can be packed into a 3D volume by spinning. Temperature can then be slightly increased to slightly melt the agarose of the agarose beads, such that the agarose beads stick to each other. In other embodiments, agarose beads can be re-suspended in small amount of additional molten agarose to adhere beads together. The attachment of beads can result in a gel matrix with spots of clonally amplified DNA (e.g., the spots of clonally amplified DNA may correspond to the beads to which the sample DNA is coupled to) or with spots of single molecules of DNA. In still other embodiments, the agarose beads may be suspended in a solution having a refractive index matching the refractive index of the agarose beads, thereby forming an optically clear suspension of beads. Prior to performing sequencing and to facilitate sequencing primer annealing, the complementary strand of the amplification products in the substrate or gel matrix can be removed, e.g., by exposure to alkali. A reaction (e.g., a sequencing reaction) may be performed on the surface of or within the agarose beads. Sequencing reagents may diffuse into the substrate or gel matrix and react or interact with nucleic acids within the substrate or gel matrix.

3D Sequencing by Droplet Generating Device using Polyacrylamide. The present disclosure provides methods allowing for 3D Sequencing using a droplet generating device and polyacrylamide (or functionalized polyacrylamide) as a substrate. A droplet generating device may have pores that enable fluid to be dropletized by centrifugation. For example, a droplet generating device may be a microcapillary array, a nozzle, or a microfluidic device (e.g., comprising a T-junction). A droplet generating device may utilize pressure (e.g., air or fluid pressure) or centrifugal force (e.g., centrifugation) to form droplets by forcing the fluid through one or more holes, pores, or channels. In such a method, the dispersion phase can contain acrylamide/bis-acrylamide and/or ammonium sulfate (or any combination thereof), in addition to reagent sets (e.g., amplification master mix) and a plurality of sample nucleic acid molecules. For amplification of sample nucleic acid molecules, one of the amplification primers (e.g., PCR primers) used in this method can be functionalized with an acrydite modification on the 5′ end such that one strand of the amplification product (e.g., PCR product) can be anchored to the acrylamide. The oil phase (e.g., oil phase of the dispersion phase) can contain one or more catalysts such as polymerization initiator compounds. Such compound can be TEMED. Upon emulsification of the mixture, the acrylamide gels can form and sample nucleic acid molecules can be encapsulated in droplets of the acrylamide gel. Droplet amplification (e.g., PCR) can be performed, during which the amplicon molecules can be attached to the gel matrix inside the droplets via the acrydite primer. Subsequent to amplification, the oil phase can be removed by washing with alcohol and detergent. During amplification and dispersion, a plurality of acrylamide beads can be formed. In some embodiments, the acrylamide beads can be formed without amplifying the nucleic acid (e.g., for single molecule sequencing). Such acrylamide beads can be packed into a 3D volume by, e.g., spinning. The beads can be held together with additional polyacrylamide solution, which can result in a gel matrix with spots of clonally amplified DNA (e.g., the spots of clonally amplified DNA may correspond to the beads to which the sample DNA is coupled to) or with spots of single molecules of DNA. Alternatively, the acrylamide beads may be suspended in a solution having a refractive index matching the refractive index of the acrylamide beads, thereby forming an optically clear suspension of beads. Prior to performing sequencing and to facilitate sequencing primer annealing, the complementary strand of the amplification products in the substrate or gel matrix can be removed, e.g., by exposure to alkali. A reaction (e.g., a sequencing reaction) may be performed on the surface of or within the acrylamide beads. Sequencing reagents may diffuse into the substrate or gel matrix and react or interact with nucleic acids within the substrate or gel matrix.

3D Sequencing by Encapsulating DNA Template Solution with Hydrogel Beads. The present disclosure provides methods allowing for 3D sequencing by encapsulating DNA template solution with hydrogel beads. In such a method, the dispersion phase can include various sets of reagents (e.g., amplificaiton master mix), a plurality of nucleic acid molecules from a sample, and a plurality of hydrogel beads (e.g., polyacrylamide beads, cross-linked agarose beads, etc.). The hydrogel beads can facilitate droplet formation upon vortexing. Alternatively, droplets may be mixed with hydrogel beads using a droplet generating device. The particles can also be conjugated with one of the sequencing primers (e.g., via 5′ acrydite modification oligo for polyacrylamide beads, via 5′ amine modification of oligo for activated agarose beads, etc.). Subsequent to amplification, clonal copies of sample nucleic acid molecules can be bound to the hydrogel beads. In some embodiments, single DNA molecules may be bound to the hydrogel beads (e.g., for single molecule sequencing). The oil phase can be removed by washing with, e.g., alcohol and/or detergent. Hydrogel beads can be packed by centrifugation, and/or held together with additional polyacrylamide solution, resulting in a gel matrix with spots of clonally amplified DNA (e.g., the spots of clonally amplified DNA can correspond to the beads to which the sample DNA is coupled to) or with spots of single molecules of DNA. Prior to performing sequence and to facilitate sequencing primer annealing, the complementary strand of the amplification products in the substrate or gel matrix can be removed, e.g., by exposure to alkali.

Thus, the methods, compositions, and kits described herein can provide a rapid, efficient, and streamlined sequencing workflow that can be used designed for analysis of a variety of samples (e.g., biological samples such as clinical samples). In particular, the aspects of 3D sequencing described herein can be combined to perform sequencing assays that can require fast turn-around, high-throughput, and easy-to-operate laboratory equipment. Some of these aspects described herein include (i) clonal amplification by digital PCR using rapid droplet generation technique; (ii) simple and fast formation of sequencing substrate by packing of droplet-generated beads (e.g., hydrogel beads) in a 3D volume; (iii) clonal amplification, sequencing substrate formation, and sequencing reaction may be carried out in the same vessel e.g., spin column or pipette tip; (iv) sequencing chemistries involving release of signal molecules may be used to facilitate long read-length (e.g., >800 bps) for nucleic acid molecules; and (v) the use of transparent 3D sequencing substrates may enable monitoring the sequencing in 3D using, e.g., lightsheet imager.

### Diseases and Conditions

The methods, compositions, and kits described herein can be useful for the analysis of nucleic acid molecules of a variety of samples, e.g., for identification of DNA mutations associated with a cancer or tumor. The cancer can comprise breast, heart, lung, small intestine, colon, spleen, kidney, bladder, head, neck, ovarian, prostate, brain, pancreatic, skin, bone, bone marrow, blood, thymus, uterine, testicular and liver tumors. The tumors can comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. The tumor/cancer can be selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, multiple myeloma, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, vipoma, and Wilm's tumor.

The methods, compositions, and kits described herein can be useful for the identification of DNA mutations associated with a disease or disorder. A disease or disorder can be angelman syndrome, canavan disease, cri du chat, cystic fibrosis, duchenne muscular dystrophy, haemochromatosis, haemophilia, neurofibromatosis, phenylketonuria, prader-willi syndrome, sickle-cell disease, 1p36 deletion syndrome, 18p deletion syndrome, 21-hydroxylase deficiency, Alpha 1-antitrypsin deficiency, AAA syndrome (achalasia-addisonianism-alacrima), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Albinism, Alexander disease, alkaptonuria, Alport syndrome, Alternating hemiplegia of childhood, Amyotrophic lateral sclerosis, Alstrom syndrome, Alzheimer's disease, Amelogenesis imperfecta, Aminolevulinic acid dehydratase deficiency porphyria, Androgen insensitivity syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Bjornstad syndrome, Bloom syndrome, Birt-Hogg-Dube syndrome, Brody myopathy, Brunner syndrome, CADASIL syndrome, CARASIL syndrome, Chronic granulomatous disorder, Campomelic dysplasiaX, Carpenter Syndrome, Cerebral dysgenesis-neuropathy-ichthyosis-keratoderma syndrome, Charcot-Marie-Tooth disease, CHARGE syndrome, Chédiak-Higashi syndrome, Cleidocranial dysostosis, Cockayne syndrome, Coffin-Lowry syndrome, Cohen syndrome, collagenopathy, types II and XI, Congenital insensitivity to pain with anhidrosis, Cowden syndrome, CPO deficiency (coproporphyria), Cranio-lenticulo-sutural dysplasia, Crohn's disease, Crouzon syndrome, Crouzonodermoskeletal syndrome (Crouzon syndrome with acanthosis nigricans), Darier's disease, Dent's disease (Genetic hypercalciuria), Denys-Drash syndrome, De Grouchy syndrome, Di George's syndrome, Distal hereditary motor neuropathies, multiple types, Edwards Syndrome, Ehlers-Danlos syndrome, Emery-Dreifuss syndrome, Erythropoietic protoporphyria, Fanconi anemia (FA), Fabry disease, factor V Leiden thrombophilia, familial adenomatous polyposis, familial dysautonomia, Feingold syndrome, FG syndrome, Friedreich's ataxia, G6PD deficiency, Galactosemia, Gaucher disease, Gillespie syndrome, Glutaric aciduria, type I and type 2, GRACILE syndrome, Griscelli syndrome, Hailey-Hailey disease, Harlequin type ichthyosis, hereditary Hemochromatosis, Hepatoerythropoietic porphyria, Hereditary coproporphyria, Hereditary hemorrhagic telangiectasia (Osler-Weber-Rendu syndrome), Hereditary Inclusion Body Myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Hyperlysinemia, hyperoxaluria, hyperphenylalaninemia, Hypoalphalipoproteinemia (Tangier disease), Hypochondrogenesis, Hypochondroplasia, Immunodeficiency, centromere instability and facial anomalies syndrome (ICF syndrome), Incontinentia pigmenti, Ischiopatellar dysplasia, Isodicentric, Jackson-Weiss syndrome, Joubert syndrome, Juvenile Primary Lateral Sclerosis (JPLS), Keloid disorder, Kniest dysplasia, Kosaki overgrowth syndrome, Krabbe disease, Kufor-Rakeb syndrome, LCAT deficiency, Lesch-Nyhan syndrome, Li-Fraumeni syndrome, Lynch Syndrome, lipoprotein lipase deficiency, Maple syrup urine disease, Marfan syndrome, Maroteaux-Lamy syndrome, McCune-Albright syndrome, McLeod syndrome, MEDNIK syndrome, Familial Mediterranean fever, Menkes disease, Methemoglobinemia, methylmalonic academia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Becker type Muscular dystrophy, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, osteogenesis imperfecta, Pantothenate kinase-associated neurodegeneration, Patau Syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic academia, Pitt-Hopkins syndrome, Polycystic kidney disease, Polycystic Ovarian Syndrome (PCOS), Porphyria, Primary ciliary dyskinesia (PCD), primary pulmonary hypertension, protein C deficiency, protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, spondyloepiphyseal dysplasia congenita (SED), Shprintzen-Goldberg syndrome, sickle cell anemia, Siderius X-linked mental retardation syndrome, Sideroblastic anemia, Sly syndrome, Smith-Lemli-Opitz syndrome, Smith Magenis Syndrome, Spinal muscular atrophySpinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous Sclerosis Complex (TSC), Turner syndrome, Usher syndrome, Variegate porphyria, von Hippel-Lindau disease, Waardenburg syndrome, Weissenbacher-Zweymüller syndrome, Williams Syndrome, Wilson disease, Woodhouse-Sakati syndrome, Wolf-Hirschhorn syndrome, Xeroderma pigmentosum, X-linked mental retardation and macroorchidism (fragile X syndrome), X-linked spinal-bulbar muscle atrophy (spinal and bulbar muscular atrophy), Xp11.22 deletion, X-linked severe combined immunodeficiency (X-SCID), X-linked sideroblastic anemia (XLSA), or Zellweger syndrome.

The methods, compositions, and kits described herein can be useful for the identification of aneuploidy. Aneuploidy can be autosomal aneuploidy or non-autosomal aneuploidy. Autosomal aneuploidy can be for chromosome 13, 18, or 21. Non-autosomal aneuploidy can be for XXX (triple X syndrome), XXXX syndrome (48, XXXX), XXXXX syndrome (49, XXXXX), or XYY syndrome.

### Digital Processing Device

The methods, compositions, and kits described herein can also include a digital processing device, or use of the same, e.g., for visualizing or monitoring 3D sequencing. The digital processing device can include one or more hardware central processing units (CPU) that carry out the device's functions. The digital processing device can further comprise an operating system configured to perform executable instructions. In some instances, the digital processing device is connected to a computer network, is connected to the Internet such that it accesses the World Wide Web, or is connected to a cloud computing infrastructure. In other instances, the digital processing device is connected to an intranet. The digital processing device can be connected to a data storage device.

In accordance with the description herein, suitable digital processing devices can include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers can include those with booklet, slate, and convertible configurations, known to those of skill in the art.

The digital processing device can include an operating system configured to perform executable instructions. The operating system can be, for example, software, including programs and data, which can manage the device's hardware and provides services for execution of applications. Those of skill in the art will recognize that suitable server operating systems can include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux. In some cases, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® P53®, Sony® P54®, Microsoft® Xbox 360®, Microsoft Xbox One, Nintendo® Wii Nintendo® Wii U®, and Ouya®.

The device can include a storage and/or memory device. The storage and/or memory device can be one or more physical apparatuses used to store data or programs on a temporary or permanent basis. In some instances, the device is volatile memory and requires power to maintain stored information. The device is non-volatile memory and retains stored information when the digital processing device is not powered. In still other instances, the non-volatile memory comprises flash memory. The non-volatile memory can comprise dynamic random-access memory (DRAM). The non-volatile memory can comprise ferroelectric random access memory (FRAM). The non-volatile memory can comprise phase-change random access memory (PRAM). The device can be a storage device including, by way of non-limiting examples, CD-ROMs, DVDs, flash memory devices, magnetic disk drives, magnetic tapes drives, optical disk drives, and cloud computing based storage. The storage and/or memory device can also be a combination of devices such as those disclosed herein.

The digital processing device can include a display to send visual information to a user. The display can be a cathode ray tube (CRT). The display can be a liquid crystal display (LCD). Alternatively, the display can be a thin film transistor liquid crystal display (TFT-LCD). The display can further be an organic light emitting diode (OLED) display. In various cases, on OLED display is a passive-matrix OLED (PMOLED) or active-matrix OLED (AMOLED) display. The display can be a plasma display. The display can be a video projector. The display can be a combination of devices such as those disclosed herein.

The digital processing device can also include an input device to receive information from a user. For example, the input device can be a keyboard. The input device can be a pointing device including, by way of non-limiting examples, a mouse, trackball, track pad, joystick, game controller, or stylus. The input device can be a touch screen or a multi-touch screen. The input device can be a microphone to capture voice or other sound input. The input device can be a video camera or other sensor to capture motion or visual input. Alternatively, the input device can be a Kinect™, Leap Motion™, or the like. In further aspects, the input device can be a combination of devices such as those disclosed herein.

### Non-Transitory Computer Readable Storage Medium

The methods disclosed herein can include one or more non-transitory computer readable storage media encoded with a program including instructions executable by the operating system of an optionally networked digital processing device A computer readable storage medium can be a tangible component of a digital processing device. A computer readable storage medium can be removable from a digital processing device. A computer readable storage medium can include, by way of non-limiting examples, CD-ROMs, DVDs, flash memory devices, solid state memory, magnetic disk drives, magnetic tape drives, optical disk drives, cloud computing systems and services, and the like. The program and instructions can be permanently, substantially permanently, semi-permanently, or non-transitorily encoded on the media.

### Computer Program

The methods disclosed herein can include at least one computer program, or use of the same. A computer program includes a sequence of instructions, executable in the digital processing device's CPU, written to perform a specified task. Computer readable instructions can be implemented as program modules, such as functions, objects, Application Programming Interfaces (APIs), data structures, and the like, that perform particular tasks or implement particular abstract data types. In light of the disclosure provided herein, those of skill in the art will recognize that a computer program, in certain embodiments, is written in various versions of various languages.

The functionality of the computer readable instructions can be combined or distributed as desired in various environments. A computer program can comprise one sequence of instructions. A computer program can comprise a plurality of sequences of instructions. A computer program can be provided from one location. A computer program can be provided from a plurality of locations. A computer program can include one or more software modules. Sometimes, a computer program can include, in part or in whole, one or more web applications, one or more mobile applications, one or more standalone applications, one or more web browser plug-ins, extensions, add-ins, or add-ons, or combinations thereof.

Computer-implemented systems can be used for the assembly of melting temperature and fluorescence data. An exemplary computer implemented system for assembly comprises a processor, wherein the processor is configured to execute the methods described herein. In an exemplary system, a processor is configured to receive a set of temperature data, receive a set of fluorescence data, assign fluorescence data to a temperature, identify the number of partitions with the same temperature and fluorescence data, and identify the target sequences in the partitions based on the temperature and fluorescence data. In another exemplary system, a processor is configured to receive a set of temperature data, receive a set of fluorescence data, assign fluorescence data to a temperature, identify the base fluorescence and temperature data relative to other base fluorescence and temperature data to determine a nucleic acid sequence, and map the nucleic acid sequence against a reference genome.

### Web Application

A computer program can include a web application. In light of the disclosure provided herein, those of skill in the art will recognize that a web application, in various aspects, utilizes one or more software frameworks and one or more database systems. A web application can be created upon a software framework such as Microsoft® .NET or Ruby on Rails (RoR). A web application can utilize one or more database systems including, by way of non-limiting examples, relational, non-relational, object oriented, associative, and XML database systems. Sometimes, suitable relational database systems can include, by way of non-limiting examples, Microsoft® SQL Server, mySQL™, and Oracle®. Those of skill in the art will also recognize that a web application, in various instances, is written in one or more versions of one or more languages. A web application can be written in one or more markup languages, presentation definition languages, client-side scripting languages, server-side coding languages, database query languages, or combinations thereof. A web application can be written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). A web application can be written to some extent in a client-side scripting language such as Asynchronous Javascript and XML (AJAX), Flash® Actionscript, Javascript, or Silverlight®. A web application can be written to some extent in a server-side coding language such as Active Server Pages (ASP), ColdFusion, Perl, Java™, JavaServer Pages (JSP), Hypertext Preprocessor (PHP), Python™, Ruby, Tcl, Smalltalk, WebDNA®, or Groovy. Sometimes, a web application can be written to some extent in a database query language such as Structured Query Language (SQL). Other times, a web application can integrate enterprise server products such as IBM® Lotus Domino®. A web application can include a media player element. A media player element can utilize one or more of many suitable multimedia technologies including, by way of non-limiting examples, Adobe® Flash®, HTML 5, Apple® QuickTime®, Microsoft® Silverlight Java™, and Unity®.

### Mobile Application

A computer program can include a mobile application provided to a mobile digital processing device. The mobile application can be provided to a mobile digital processing device at the time it is manufactured. In other cases, the mobile application is provided to a mobile digital processing device via the computer network described herein.

In view of the disclosure provided herein, a mobile application can be created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

A computer program can include a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. A computer program can include one or more executable complied applications.

### Web Browser Plug-In

The computer program can include a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) can be software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The methods disclosed herein can include software, server, and/or database modules, or use of the same. In view of the disclosure provided herein, software modules can be created by techniques known to those of skill in the art using machines, software, and languages known to the art. The software modules disclosed herein can be implemented in a multitude of ways. A software module can comprise a file, a section of code, a programming object, a programming structure, or combinations thereof. A software module can comprise a plurality of files, a plurality of sections of code, a plurality of programming objects, a plurality of programming structures, or combinations thereof. The one or more software modules can comprise, by way of non-limiting examples, a web application, a mobile application, and a standalone application. Software modules can be in one computer program or application. Software modules can be in more than one computer program or application. Software modules can be hosted on one machine. Software modules can be hosted on more than one machine. Software modules can be hosted on cloud computing platforms. Software modules can be hosted on one or more machines in one location. Software modules are hosted on one or more machines in more than one location.

### Databases

The methods disclosed herein can include one or more databases, or use of the same. In view of the disclosure provided herein, those of skill in the art will recognize that many databases are suitable for storage and retrieval of analytical information described elsewhere herein. Suitable databases can include, by way of non-limiting examples, relational databases, non-relational databases, object oriented databases, object databases, entity-relationship model databases, associative databases, and XML databases. A database can be internet-based. A database can be web-based. A database can be cloud computing-based. Alternatively, a database can be based on one or more local computer storage devices.

### Services

Methods described herein can further be performed as a service. For example, a service provider can obtain a sample that a customer wishes to analyze. The service provider can then encode the sample to be analyzed by any of the methods described herein, performs the analysis and provides a report to the customer. The customer can also perform the analysis and provide the results to the service provider for decoding. The service provider can then provide the decoded results to the customer. The customer can received encoded analysis of the samples from the provider and can decode the results by interacting with software installed locally (at the customer's location) or remotely (e.g., on a server reachable through a network). The software can generate a report and transmit the report to the costumer. Exemplary customers include clinical laboratories, hospitals, industrial manufacturers, and the like. Sometimes, a customer or party can be any suitable customer or party with a need or desire to use the methods provided herein.

### Server

The methods provided herein can be processed on a server or a computer server. The server can include a central processing unit (CPU, also “processor”) which can be a single core processor, a multi core processor, or plurality of processors for parallel processing. A processor used as part of a control assembly can be a microprocessor. The server can also include memory (e.g., random access memory, read-only memory, flash memory); electronic storage unit (e.g., hard disk); communications interface (e.g., network adaptor) for communicating with one or more other systems; and peripheral devices which includes cache, other memory, data storage, and/or electronic display adaptors. The memory, storage unit, interface, and peripheral devices can be in communication with the processor through a communications bus (solid lines), such as a motherboard. The storage unit can be a data storage unit for storing data. The server can be operatively coupled to a computer network (“network”) with the aid of the communications interface. A processor with the aid of additional hardware can also be operatively coupled to a network. The network can be the Internet, an intranet and/or an extranet, an intranet and/or extranet that is in communication with the Internet, a telecommunication or data network. The network with the aid of the server, can implement a peer-to-peer network, which can enable devices coupled to the server to behave as a client or a server. The server can be capable of transmitting and receiving computer-readable instructions (e.g., device/system operation protocols or parameters) or data (e.g., sensor measurements, raw data obtained from detecting metabolites, analysis of raw data obtained from detecting metabolites, interpretation of raw data obtained from detecting metabolites, etc.) via electronic signals transported through the network. Moreover, a network can be used, for example, to transmit or receive data across an international border.

The server can be in communication with one or more output devices such as a display or printer, and/or with one or more input devices such as, for example, a keyboard, mouse, or joystick. The display can be a touch screen display, in which case it functions as both a display device and an input device. Different and/or additional input devices can be present such an enunciator, a speaker, or a microphone. The server can use any one of a variety of operating systems, such as for example, any one of several versions of Windows®, or of MacOS®, or of Unix®, or of Linux®.

The storage unit can store files or data associated with the operation of a device, systems or methods described herein.

The server can communicate with one or more remote computer systems through the network. The one or more remote computer systems can include, for example, personal computers, laptops, tablets, telephones, Smart phones, or personal digital assistants.

A control assembly can include a single server. The system can include multiple servers in communication with one another through an intranet, extranet and/or the Internet.

The server can be adapted to store device operation parameters, protocols, methods described herein, and other information of potential relevance. Such information can be stored on the storage unit or the server and such data is transmitted through a network.

### EXAMPLES

These examples are provided for illustrative purposes only and not to limit the scope of the claims provided herein.

### Example 1

### Generation of a 3D Sequencing Substrate

This example demonstrates the generation of a three-dimensional (3D) sequence substrate that may be used for analysis of sample nucleic acid molecules (e.g., nucleic acid molecules of a biological/clinical sample).

The nucleic acids molecules (in this case: purified genomic DNA) of a biological sample (e.g., a clinical sample of a patient) are clonally amplified using rapid droplet digital polymerase chain reaction (PCR) techniques or isothermal amplification techniques. In a first technique, emulsion droplets are generated using a droplet generating device (e.g., a microcapillary array or a microfluidic device) and centrifugation or pressure. A droplet generative device typical for forming droplets (e.g., a nozzle) may be used. The droplets of amplification mixture containing amplification reagents, nucleic acids, and a component that can be solidified after amplification (e.g., agarose or polyacrylamide) are formed by using the droplet generating device. Droplets are formed such that at most one nucleic acid molecule is occupied in one partition or droplet. Droplets are generated and amplification is performed as described in EXAMPLE 2 or in EXAMPLE 3. The droplets are collected in a vessel and alcohol or detergent is used to break the emulsion, releasing the beads. The beads are then packed by centrifugation, forming a 3D substrate.

In a second technique, emulsion droplets are generated by vortexing with hydrogel beads. Droplets are formed such that at most one nucleic acid molecule is occupied in one partition or droplet. The emulsion mixture is comprised of amplification reagents and polymer beads, e.g., agarose beads, hydrogel beads, or polyacrylamide. beads Subsequent to the completion of emulsion amplification, the mixture is transferred to a vessel, such as a spin column or a pipette tip. The oil phase is removed using ethanol or detergent and the formed polymer beads comprising the nucleic acid molecules and amplicons thereof are packed into a 3D volume by centrifugation, forming a 3D sequencing substrate.

The substrate that is used in this example is optically clear. The 3D sequencing substrate is rendered optically clear by adding additional polymer solution to form an optically clear gel matrix, heating the substrate to slightly melt the polymer beads such that the polymer beads stick together and form an optically clear substrate, or suspending the polymer beads in a solution with a refractive index matching the refractive index of the polymer beads to form an optically clear suspension of beads. Thus, imaging techniques such as lightsheet imaging is used to image the substrate volume. TABLE 1 below shows an exemplary number of positive droplets (or reads) that are fitted into a volume of 100 μl, assuming 10% of the droplets are positive. For 15 μm droplets, ˜4 million reads are attained. For 10 μm droplets, ˜14 million reads are achieved.

The resulting numbers of reads are sufficient to allow clinical assays (e.g., targeted panels, shallow whole genome for non-invasive prenatal testing (NIPT), etc.) of a single sample.

This example demonstrates that the herein described methods, compositions, and kits are used to generate 3D sequencing substrates comprising amplified sample nucleic acid molecules that are analyzed using 3D sequencing as described herein. High-throughput, easy-to-use and rapid sequence analysis of nucleic acid molecules, which is particularly relevant for analysis of clinical samples, is attained by using the method as described herein.

TABLE 1
Packing results of droplets in a 3D vessel

| | 30 μm | 25 μm | 20 μm | 15 μm | 10 μm | 5 μm | 1 μm |
|---|---|---|---|---|---|---|---|
| Droplet | 3.00 × 10⁻⁵ | 2.50 × 10⁻⁵ | 2.00 × 10⁻⁵ | 1.50 × 10⁻⁵ | 1.00 × 10⁻⁵ | 6.00 × 10⁻⁶ | 1.00 × 10⁻⁶ |
| Diameter (m) | | | | | | | |
| Volume (L) | 1.41 × 10⁻¹¹ | 8.18 × 10⁻¹² | 4.19 × 10⁻¹² | 1.77 × 10⁻¹² | 5.24 × 10⁻¹³ | 6.54 × 10⁻¹⁴ | 5.24 × 10⁻¹⁶ |
| Volume (pL) | 14.1 | 8.18 | 4.19 | 1.77 | 0.524 | 6.54 × 10⁻² | 5.24 × 10⁻⁴ |
| Total Volume in | 1.00 × 10⁻⁴ | 1.00 × 10⁻⁴ | 1.00 × 10⁻⁴ | 1.00 × 10⁻⁴ | 1.00 × 10⁻⁴ | 1.00 × 10⁻⁴ | 1.00 × 10⁻⁴ |
| Vessel (L) | | | | | | | |
| Volume of Hex | 7.40 × 10⁻⁵ | 7.40 × 10⁻⁵ | 7.40 × 10⁻⁵ | 7.40 × 10⁻⁵ | 7.40 × 10⁻⁵ | 7.40 × 10⁻⁵ | 7.40 × 10⁻⁵ |
| Closed Pack (L) | | | | | | | |
| Number of | 5.23 × 10⁶ | 9.05 × 10⁶ | 1.77 × 10⁷ | 4.19 × 10⁷ | 1.41 × 10⁸ | 1.13 × 10⁹ | 1.41 × 10¹¹ |
| Possible | | | | | | | |
| Droplets in | | | | | | | |
| Vessel | | | | | | | |
| Number of | 5.23 × 10⁵ | 9.05 × 10⁵ | 1.77 × 10⁶ | 4.19 × 10⁶ | 1.41 × 10⁷ | 1.13 × 10⁸ | 1.41 × 10¹⁰ |
| Positive | | | | | | | |
| Droplets, | | | | | | | |
| Assuming 10% | | | | | | | |
| Occupancy | | | | | | | |

### Example 2

### 3D Sequencing and Droplet Generation by Microcapillary Array Using Agarose

This example demonstrates 3D sequencing and droplet generation using a microcapillary array and agarose as substrate.

For droplet generation, the dispersion phase is comprised of molten agarose in addition to PCR master mix and sample nucleic acid molecules. PCR in the emulsion droplets is performed. At PCR cycling temperatures (e.g., >50° C.), agarose is maintained as a liquid, allowing efficient distribution of material and reagents within the mixture. A PCR primers is used that carries an amine modification on the 5′ end such that one strand of the PCR product is anchored to agarose. After PCR, the temperature is lowered and the agarose is solidified. The oil phase is removed by washing with alcohol and detergent. Agarose beads are then packed by spinning. Temperature is increased slightly to melt agarose, such that agarose beads stick to each other. Alternatively, agarose beads are re-suspended in small amount of additional molten agarose to adhere beads together. The result is a gel matrix with spots of clonally amplified sample/template DNA. To facilitate sequencing primer annealing, the complementary strand of the PCR product is removed by exposure to alkali.

This example demonstrates efficient generation of 3D sequencing substrates that are used for the analysis of biological samples.

### Example 3

### 3D Sequencing by Microcapillary Array Using Polyacrylamide

This example demonstrates 3D sequencing using a microcapillary array and polyacrylamide as substrate.

For droplet/emulsion PCR, the dispersion phase is comprised of acrylamide/bis-acrylamide and ammonium sulfate, in addition to PCR master mix and sample nucleic acid molecules. A PCR primer is used that carries an acrydite modification on the 5′ end such that one strand of the PCR product is anchored to the acrylamide inside the droplet. The oil phase also is comprised of TEMED as a polymerization initiator. Upon emulsification, the acrylamide gels and template DNA are encapsulated in the gel matrix inside the droplet. Droplet PCR is performed, during which the amplicon molecules are attached to the gel matrix via the acrydite primer. After PCR, the oil phase is removed by washing with alcohol and/or detergent. Acrylamide beads are packed into a 3D volume by spinning. Alternatively, the beads are held together with additional polyacrylamide solution. End result is a gel matrix with spots of clonally amplified DNA. To facilitate sequencing primer annealing, the complementary strand of the PCR product is removed by exposure to alkali.

This example demonstrates efficient generation of 3D sequencing substrates that are used for the analysis of biological samples.

### Example 4

### 3D Sequencing by Vortexing DNA Template Solution with Hydrogel Beads

This example demonstrates 3D sequencing by vortexing DNA template solution with hydrogel beads.

For droplet/emulsion PCR, the dispersion phase is comprised of PCR master mix, sample nucleic acid molecules, and hydrogel beads (e.g., polyacrylamide or cross-linked agarose). Upon vortexing of the mixture, droplet formation of the hydrogel beads is facilitated. The particles are also conjugated with one of the sequencing primers via a 5′ acrydite modification oligonucleotide for polyacrylamide beads, or via 5′ amine modification of oligo for activated agarose beads. Upon completion of PCR, clonal copies are bound to the hydrogel beads. The oil phase is then removed by washing with alcohol and detergent. Hydrogel beads are packed into a 3D volume by centrifugation, and/or are held together with additional polyacrylamide solution. The result is a gel matrix with spots of clonally amplified DNA. To facilitate sequencing primer annealing, the complementary strand of the PCR product is removed by exposure to heat or alkali.

This example demonstrates efficient generation of 3D sequencing substrates that are used for the analysis of biological samples.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of forming a three dimensional (3D) sequencing substrate comprising:
(a) amplifying a plurality of nucleic acid molecules from a sample in a plurality of partitions, wherein a partition of the plurality of partitions comprises a nucleic acid molecule from the plurality of nucleic acid molecules and a substrate, and wherein amplification couples the nucleic acid molecule from the plurality of nucleic acid molecules or an amplicon thereof to the substrate; and
(b) forming a three dimensional (3D) sequencing substrate from the plurality of partitions.

2. A method of forming a three dimensional sequencing substrate comprising:
(a) distributing a plurality of nucleic acid molecules of a sample into a plurality of partitions, wherein a partition of the plurality of partitions comprises a nucleic acid molecule of the plurality of nucleic acid molecules and a substrate of a plurality of substrates;
(b) coupling the nucleic acid molecule of the plurality of nucleic acid molecules to the substrate of the plurality of substrates in the partition of the plurality of partitions to form a substrate conjugate of a plurality of substrate conjugates, thereby generating the plurality of substrate conjugates in the plurality of partitions; and
(c) forming a three dimensional sequencing substrate from the plurality of partitions.

3. A method of sequencing a plurality of nucleic acid molecules of a sample, the method comprising:
(a) forming a three dimensional (3D) sequencing substrate from a plurality of partitions, wherein a partition of the plurality of partitions comprises a substrate conjugate, and the substrate conjugate comprises a nucleic acid molecule of the plurality of nucleic acid molecules of the sample coupled to a substrate; and
(b) sequencing the plurality of nucleic acid molecules in the three dimensional sequencing substrate.

4. A method of sequencing a plurality of nucleic acid molecules of a sample, the method comprising:
(a) distributing the plurality of nucleic acid molecules of the sample into a plurality of partitions, wherein a partition of the plurality of partitions comprises a nucleic acid molecule of the plurality of nucleic acid molecules and a substrate of a plurality of substrates;
(b) coupling the nucleic acid molecule of the plurality of nucleic acid molecules to the substrate of the plurality of substrates in the partition of the plurality of partitions to form a substrate conjugate of a plurality of substrate conjugates, thereby generating the plurality of substrate conjugates in the plurality of partitions;
(c) forming a three dimensional sequencing substrate from the plurality of partitions; and
(d) sequencing the plurality of nucleic acid molecules in the three dimensional (3D) sequencing substrate.

5. A method of identifying a plurality of nucleic acid molecules of a sample, the method comprising:
(a) coupling the plurality of nucleic acid molecules to a substrate to produce a plurality of coupled nucleic acid molecules;
(b) partitioning the plurality of coupled nucleic acid molecules into a plurality of partitions such that each partition comprises a nucleic acid molecule coupled to the substrate;
(c) forming a three dimensional (3D) sequencing substrate from the plurality of partitions; and
(d) sequencing the plurality of coupled nucleic acid molecules, thereby identifying the plurality of nucleic acid molecules of the sample.

6. The method of any one of claims 2-5, further comprising, prior to (a), (b), or (c), amplifying the plurality of nucleic acid molecules in the plurality of partitions.

7. The method of any one of claims 1-6, wherein amplification comprises thermal cycling amplification or isothermal amplification.

8. The method of any one of claims 1-7, wherein the nucleic acid molecule or an amplicon thereof is coupled to the substrate using bioconjugation chemistry or click chemistry.

9. The method of any one of claims 1-8, wherein the nucleic acid molecule or an amplicon thereof is coupled to the substrate using a PCR primer comprising a modification at the 5′-end.

10. The method of claim 9, wherein the modification at the 5′-end comprises an acrydite moiety.

11. The method of any one of claims 1-10, wherein the plurality of partitions comprises a plurality of droplets.

12. The method of claim 11, wherein the plurality of droplets comprises a plurality of emulsion droplets.

13. The method of any one of claims 1-12, wherein the substrate comprises a polymer.

14. The method of any one of claims 1-13, wherein the polymer comprises an agarose, a polyacrylamide, a UV-curable polymer, a PEG based hydrogel, or a combination thereof.

15. The method of any one of claims 1-14, wherein the substrate conjugate is an emulsion bead-nucleic acid conjugate, a polymer bead-nucleic acid conjugate, or a combination thereof.

16. The method of any one of claims 1-15, wherein the plurality of partitions are attached to each other, thereby forming the 3D sequencing substrate.

17. The method of claim 16, wherein plurality of partitions are attached to each other by addition of substrate to the plurality of partitions, by an elevation of temperature, or a combination thereof.

18. The method of claim 17, wherein the 3D sequencing substrate is a gel matrix.

19. The method of any one of claims 3-18, wherein the sequencing is conducted in a vessel.

20. The method of claim 19, wherein the vessel is a sphere, a cylinder, a cube, a cone, a hexagon, a prism, or any combination or variation thereof.

21. The method of any one of claims 19-20, wherein the vessel is a tube, a syringe, a micro-container, a spin column, a flow cell, or a combination thereof.

22. The method of any one of claims 1-21, wherein the 3D sequencing substrate has the same shape as the vessel.

23. The method of any one of claims 1-22, wherein the 3D sequencing substrate is formed by centrifugation.

24. The method of any one of claims 1-23, wherein the 3D sequencing substrate has a volume from about 1 μL to about 1000 μL.

25. The method of any one of claims 1-24, wherein the 3D sequencing substrate comprises from about 10³ to about 10¹⁵ partitions.

26. The method of any one of claims 1-25, wherein the plurality of partitions of the 3D sequencing substrate assemble in a cubic closest packed unit cell.

27. The method of any one of claims 1-26, wherein each partition of a plurality of partitions has an average diameter of about 1 μm to about 50 μm.

28. The method of any one of claims 3-27, wherein sequencing is performed inside the 3D sequencing substrate.

29. The method of any one of claims 1-28, wherein the 3D sequencing substrate is transparent.

30. The method of any one of claims 1-29, wherein the sequencing reaction in the 3D sequencing substrate is monitored in 3D using 3D imaging.

31. The method of any one of claims 1-30, wherein 3D imaging comprises confocal microscopy, super-resolution confocal microscopy, multiphoton microscopy, or lightsheet microscopy, or a combination thereof.

32. The method of any one of claims 1-31, wherein the substrate conjugates in the 3D sequencing substrate are detected via 3D imaging as spots of nucleic acid molecules.

33. The method of any one of claims 1-32, wherein one or more nucleic acid molecules of the plurality of nucleic acid molecules are detected in no more than about 50%, no more than about 10%, no more than about 5%, or no more than about 1% of partitions of the plurality of partitions of the 3D sequencing substrate.

34. The method of any one of claims 3-33, wherein sequencing comprises pyrosequencing, sequencing by synthesis, sequencing by ligation, sequencing by hybridization, sequencing by degradation, sequencing by detection of local pH changes, sequencing by denaturation, or any combination thereof.

35. A composition comprising:
(a) a three dimensional (3D) sequencing substrate;
(b) a plurality of nucleic acid molecules; and
(c) sequencing reagents.

36. The composition claim 35, wherein the 3D sequencing substrate comprises a plurality of partitions.

37. The composition of claim 36, wherein a partition of the plurality of partitions comprises a nucleic acid molecule of the plurality of nucleic acid molecules.

38. The composition of any one of claims 35-37, wherein the nucleic acid molecule of the plurality of nucleic acid molecules is coupled to the substrate inside the partition.

39. The composition of any one of claims 35-38, wherein the plurality of partitions is a plurality of droplets

40. The composition of any one of claims 35-39, wherein the substrate comprises a polymer.

41. The composition of claim 40, wherein the polymer comprises an agarose, a polyacrylamide, a UV-curable polymer, a PEG based hydrogel, or a combination thereof.

42. The composition of any one of claims 35-41, wherein the nucleic acid molecule is coupled to the substrate using bioconjugation chemistry or click chemistry.

43. The composition of any one of claims 35-42, wherein the nucleic acid molecule or an amplicon thereof is coupled to the substrate using a PCR primer comprising a modification at the 5′-end.

44. The composition of claim 43, wherein the modification at the 5′-end comprises an acrydite moiety.

45. The composition of any one of claims 35-44, wherein the plurality of partitions forming the 3D sequencing substrate are attached to each other.

46. The composition of claim 45, wherein the plurality of partitions are attached to each other by addition of substrate to the plurality of partitions, by an elevation of temperature, or a combination thereof.

47. A kit for nucleic acid sequence identification of a sample comprising:
(a) substrate reagents for forming a three dimensional (3D) sequencing substrate;
(b) amplification reagents;
(c) sequencing reagents; and
(d) instructions that direct a user to use the substrate reagents, the amplification reagents, and the sequencing reagents for nucleic acid sequence identification of the sample in the 3D sequencing substrate.

48. The kit of claim 47, wherein the 3D sequencing substrate has the same shape as the part of the vessel comprising the 3D sequencing substrate.

49. The kit of any one of claims 47-48, further comprising a vessel in which to form the 3D sequencing substrate.

50. The kit of claim 49, wherein the vessel is a tube, a syringe, a micro-container, a spin column, a flow cell, or a combination thereof.

51. The kit of any one of claims 47-50, further comprising a plurality of nucleic acid molecules of the sample, and wherein the plurality of nucleic acid molecules is coupled to the 3D sequencing substrate, thereby forming a plurality of substrate conjugates in the 3D sequencing substrate.

52. The kit of any one of claims 47-51, wherein the substrate reagents comprise agarose, a polymer, or a hydrogel.

53. The kit of any one of claims 47-52, wherein the 3D sequencing substrate is transparent.

54. The kit of any one of claims 47-53, wherein the sequencing reaction using the sequencing reagents in the 3D sequencing substrate is monitored in 3D using 3D imaging.

55. The kit of any one of claims 47-54, wherein 3D imaging comprises confocal microscopy, super-resolution confocal microscopy, multiphoton microscopy, or lightsheet microscopy, or a combination thereof.

56. The kit of any one of claims 47-55, wherein the substrate conjugates in the 3D sequencing substrate are detected as spots of nucleic acid molecules.

57. The kit of any one of claims 47-56, wherein the plurality of substrate conjugates is a plurality of emulsion bead-nucleic acid conjugates, a plurality of polymer bead-nucleic acid conjugates, or a combination thereof.

58. The kit of any one of claims 47-57, wherein the 3D sequencing substrate has a volume of about 1 μL to about 1000 μL.

59. The kit of any one of claims 47-58, wherein the sequencing comprises pyrosequencing, sequencing by synthesis, sequencing by ligation, sequencing by hybridization, sequencing by degradation, sequencing by detection of local pH changes, sequencing by denaturation, or any combination thereof.
